(19) United States
(12) Patent Application Publication
(10) Pub. No.: US 2008/0195218 A1
(43) Pub. Date: Aug. 14, 2008

(54) Prosthetic implant for use without bone cement

(75) Inventor: Eric Jones, Limerick (IE)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Benoist Girard SAS, Herouville-saint-clair Cedex (FR)

(21) Appl. No.: 11/811,890

(22) Filed: Jun. 11, 2007

(30) Foreign Application Priority Data
Feb. 14, 2007 (GB) 0702864.0

(51) Int. Cl. A61F 2/38; A61F 2/30; A61F 2/32

(52) U.S. Cl. 623/20.36; 623/22.11; 623/23.44; 623/23.46

(57) ABSTRACT

A prosthesis for implantation in a femur has a metallic stem portion having a smooth outer surface and a distal tip. A polymethylmethacrylate (PMMA) sleeve surrounds and is in contact with the stem portion. The sleeve has a cavity for receiving the stem tip and a bottom of said cavity spaced from the tip. A metal sheath has a cavity for receiving the sleeve. The metal sheath has a porous outer tissue ingrowth surface and a roughened inner surface. The PMMA sleeve is bonded to the inner surface of the metal sheath with a PMMA bone cement. The stem portion, sleeve and metal sheath all taper inwardly moving from a proximal stem portion to a distal stem portion.

### BACKGROUND OF THE INVENTION

This invention relates to a prosthetic implant, of any kind, which has any attachment portion for insertion into or attachment to a patient's bone without bone cement. Thus the invention can be applied, for example, to a femoral prosthesis, a tibial component of a total knee prosthesis or a hip cup.

The term “bone ingrowth” will be used herein to indicate a surface onto which bone can grow, for example a roughened surface. The term “bone ingrowth” will be used herein to indicate a surface in which bone can grow inwardly, for example a porous surface.

It is known from, for example, U.S. Pat. No. 5,665,121 to use a sheath made from a synthetic plastics material which could have a roughened outer surface to promote bone ongrowth over the stem of a femoral prosthesis for use without bone cement. It is also known from U.S. Pat. No. 4,650,489 to provide a femoral prosthesis with an outer sheath made of stainless steel or titanium which encloses the stem but is separated from it by a layer of elastomeric material such as silicon or a butyl rubber. The inner surface of the metallic sheath and the outer surface of the stem are indented to retain the elastomeric material in place and prevent movement between the stem and the outer metallic sheath. The distal end of the stem is located in a closed cavity filled with an air/gas below the elastomeric material to allow the stem to displace slightly under shock due to the resilience of the elastomeric material but there is no provision to allow the stem to subside downwardly within the sheath after insertion.

The present invention is intended to provide a construction which has the advantage over those referred to in the earlier documents in that it allows a sheath to be proximally loaded by arranging the outer surface to be metallic and encourage bone ingrowth, and for the distal part of the sheath to encourage bone ongrowth or, preferably, only a very minor ingrowth or none at all. With this arrangement the loading on the distal end of the sleeve is reduced in relation to the proximal loading, which has been found to be desirable.

### SUMMARY OF THE INVENTION

According to the present invention a prosthetic implant has an attachment portion for insertion into or attachment to a patient's bone without bone cement and the outer surface of the attachment portion which is to be attached to said bone has a first layer made of synthetic resin material over which is secured a second layer made of a metallic material, the proximal portion of the second layer being porous or roughened to encourage bone ingrowth or ongrowth and a distal portion thereof being less porous, substantially non-porous or less roughened in relation to the first portion, or smooth. This type of construction allows for the desirable proximal loading capability.

Thus, the outer surface of the metallic layer can, for example, have an interconnected porosity where bone ingrowth is required on the proximal portion and a roughened surface where bone ongrowth is required on the outer distal surface of the metallic layer or, preferably, this portion may be relatively smooth. In some embodiments the required proximal locking can be achieved by a roughened surface on the proximal portion and smooth or less roughened on the distal portion.

The first and second layers can be applied in situ to the attachment portion, for example the plastic sheath is preferably cast into position on the attachment portion with which it is to be used and this can be temporarily fitted with a mechanism to create the distal void.

Alternatively the first and second layers can be preformed as a separate sheath for attachment to the attachment portion. Again, the plastic inner layer can be cast on the intended femoral implant or, alternatively, the inner layer can be molded into the outer metallic layer.

A sheath can also be made which is not preferentially matched with the intended femoral implant, for example, the sheath could be of a standard dimension which could be used with existing implants. The second metallic material layer can be made from titanium, titanium alloy or any other suitably bio-compatible metal which contacts directly with the bore. The metal can be in the form of a preformed shape or can be formed by metallic sputtering or, for example, forming by a laser melting process.

The layer melting process can, for example, be as set forth in U.S. Patent Publications 20040191106 entitled, “Laser-Produced Porous Surface”, 20060147332 entitled, “Gradient Porous Implant”, and U.S. patent application Ser. Nos. 11/295,008 entitled, “Laser-Produced Porous Surface”, and 60/755,260 filed Dec. 30, 2005 entitled “Laser-Produced Implants”, all the disclosures of which are hereby incorporated by reference herein. As discussed in U.S. Patent Publication 20040191106, the metal structure may be constructed using a selective laser melting or sintering process, which hereby grows the structure in a layer by layer process. In the alternative, the metal structure may be built using an alternate process described in U.S. Patent Publication 2004019116 wherein the intermediate portion acts as a base or substrate on which the polymer engaging portion and bone ingrowth portion are built thereon, also in a layer-by-layer fashion. Additional techniques for constructing the metal lattice may also be employed such as that disclosed in U.S. Patent Publication 20030153981 entitled, “Porous Metallic Scaffold for Tissue Ingrowth”, the disclosure of which is hereby incorporated by reference herein, as well as additional methods known to those in the art such as that disclosed in U.S. Patent Publications 20060228247 and 20060002810, the disclosures of which is hereby incorporated by reference herein.

Preferably, the synthetic resin material is polymethylmethacrylate (PMMA). Preferably there is engagement or interlock between the first and second layers, for example by providing a roughened surface on inner surface of the second layer.

The distal end of the first and second layers can be formed as a cup, the inner surface of which is spaced away from the distal end of the attachment portion to provide a distal void when initially located in position to accept subsequent movement between the first layer and the attachment portion after fitting. This movement allows the stem to subside to its natural position after initial weight bearing of the implanted device.

The thickness of the wall formed by the two layers can be between 1 mm and 3 mm and is preferably between 1.8 mm and 2.5 mm.

As used herein when referring to bones or other parts of the body, the term “proximal” means close to the heart and the term “distal” means more distant from the heart. The term “inferior” means toward the feet and the term “superior” means toward the head. The term “anterior” means toward the front part or the face and the term “posterior” means toward the back of the body. The term “medial” means toward the midline of the body and the term “lateral” means away from the midline of the body.

Various aspects of the invention are achieved by a prosthesis for implantation in a femur which prosthesis has a metallic stem portion with a smooth outer surface. Preferably the stem has a polished outer surface and a generally rectangular cross section. The thickness of the cross section in both the anterior-posterior and medial-lateral directions tapers on moving proximally to distally. The stem portion terminates in a tip at its distal end. The prosthesis further includes a polymethylmethacrylate (PMMA) sleeve surrounding and in contact with the stem portion outer surface. The sleeve has a cavity for receiving the stem tip with a bottom of the cavity in the sleeve spaced from the tip to allow migration of the stem distally within the sleeve. A metal sheath having a cavity is provided with the cavity being shaped for receiving the sleeve. The metal sheath has a porous outer tissue ingrowth surface which is more porous in the proximal area of the sheath than in the distal area. In a preferred embodiment the distal area is solid i.e. non-porous. The inner surface of the metal sheath may be roughened to enhance the sheath being bonded to the PMMA sleeve outer surface. Preferably this bonding takes place with an additional PMMA bone cement. This assembly can either be accomplished at the factory or in the operating room. In general, the combined thickness of the sleeve/metal sheath combination is between 1 and 3 mm.

A method for forming the prosthetic implant is also disclosed which includes providing a prosthetic implant having a tapered metal stem portion in a roll with a neck portion. The neck portion may include a trunion for receiving a modular prosthetic femoral head. The PMMA sleeve may then be attached to the stem portion, which stem portion may be highly polished, by dipping the stem into a liquid PMMA bath or by spraying the PMMA onto the outer surface of the stem. Once dried the metal sheath may be applied to the PMMA coating by either buttering a metal, such as titanium, onto the PMMA surface or by a selective laser sintering process. Alternately, the sheath can be formed separately and slid onto the stem in a distal to proximal direction and, likewise, the sheath can be preformed and bonded to the outer surface of the sleeve by using additional PMMA bone cement.

### BRIEF DESCRIPTION OF THE DRAWINGS

The invention can be performed in various ways but one embodiment will now be described by way of example with reference to the accompanying drawings in which:

FIG. 1 is a part-cross-sectional side elevation of a femoral prosthesis embodying the invention; and

FIG. 2 is a cross-sectional plan view on the line II-II shown in FIG. 1.

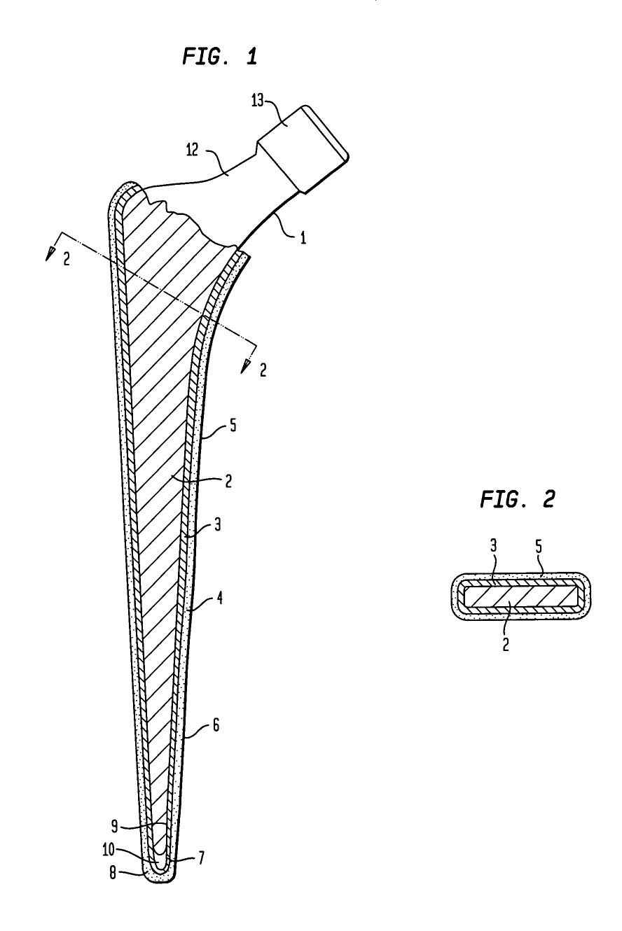

### DETAILED DESCRIPTION

As shown in the drawings, the invention is applied to a prosthetic implant in the form of a femoral prosthesis 1 which has an attachment portion for insertion into or attachment to a patient's bone without bone cement in the form of a stem 2. The outer surface of the stem 2 is smooth and may be polished and tapered on moving proximal to distal on the stem. The stem 2 has a first layer 3 made of a synthetic resin material which in this example is polymethylmethacrylate.

A second layer made of a metallic material 4 is applied over the first layer 3 and the proximal part 5 of the second layer 4 is porous. The distal portion 6 (in the diaphysis of the femur) of second metallic layer 4 is less porous or substantially non-porous in relation to proximal portion (in the epiphysis and metaphysic of the femur) 5.

With this construction proximal part 5 of the second layer 4 which is porous provides for bone ingrowth and distal portion 6 can either be less porous or substantially non-porous by providing it as a roughened surface to allow for bone ongrowth. Thus the maximum bone attachment is provided at the proximal end where the stem is proximally loaded. In the area where no distal locking is required the outer surface of distal portion 6 could be relatively smooth.

Alternatively, proximal part 5 of second layer 4 can be roughened rather than being porous to allow for bone ongrowth and distal portion 6 can be less roughened or substantially smooth to provide the same effect. The second layer 4 can be made from titanium, titanium alloy or any other bio-compatible metal which contacts directly with the bone.

The distal end 7 of first layer 3 and second layer 4 are formed as a cup 8, the inner surface of which is spaced away from the distal end 9 of stem 2 to provide a void 10 when initially located in position. This void 10 can accept subsequent movement between first layer 3 and the tem after fitting.

The thickness of the wall formed by layer 3 and 4 can be between 1 mm and 3 mm and is preferably between 1.8 mm and 2.5 mm. The first and second layers 3 and 4 can be applied in situ to stem 2, for example, the plastic first layer can be cast into position on the stem with which it is to be used and this can then be temporarily fitted with a mechanism to create the distal void.

Alternatively, the first and second layers can be preformed as a separate sheath for attachment to the stem. Again, plastic inner layer 3 can be cast on the stem or, alternatively, this inner layer can be molded into the outer metallic second layer 4.

A sheath of this type can be made which is not preferentially matched with the intended femoral implant, for example, a sheath could be of a standard dimension which could be used with the existing implants.

During construction a second layer 3 can be applied to a stem 2 by dipping or a spraying process and the third metallic layer 4 can be applied, for example, by sputtering or any other layering process or it can be made by a laser melting process such as disclosed in U.S. Publications 20040191106 and 20060147332.

This will still allow the stem to move downwardly after fitting provided the surface of the stem has a suitable smooth surface. Alternatively the first and second layers 3 and 4 can be preformed as a sheath by forming on a suitable former, for example, the stem with which it is intended to be matched.

If the proximal portion of metallic layer 4 is porous it allows for boney ingrowth and firm fixation of the assembly into the bone and the solid or less porous distal portion 6 allows for bone ongrowth this portion being fitted as an interference fit between the sheath and the surrounding bone when installed.

In the construction shown the femoral prosthesis has a neck 12 and a tapered spigot or trunion 13 to receive a prosthetic head bearing ball (not shown) of well-known type.

In the example described above the invention is applied to a femoral prosthesis but it can be equally well applied to any other prosthesis which has an attachment portion which is for insertion into or attachment to a patient's bone without bone cement.

If desired a layer of bio-active material, such as hydroxyapatite (not shown) can be applied to the outer surface of second metallic layer 4 to encourage bone ingrowth or ongrowth.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthesis for implantation in a femur comprising:
a metallic stem portion having a smooth outer surface and a distal tip; and
a polymethylmethacrylate (PMMA) sleeve surrounding and in contact with the stem portion, the sleeve having a cavity for receiving the stem tip, a bottom of said cavity spaced from the tip; and
a metal sheath having a cavity for receiving the sleeve, the metal sheath having a proximal porous outer tissue ingrowth surface and a roughened inner surface, the PMMA sleeve bonded to the inner surface of the metal sheath with a PMMA bone cement.

2. The prosthesis as set forth in claim 1 wherein the stem portion, sleeve and metal sheath taper inwardly moving from a proximal stem portion to a distal stem portion.

3. The prosthesis as set forth in claim 2 wherein the stem portion is polished.

4. The prosthesis as set forth in claim 1 wherein a proximal portion of the metal sheath has a porous outer tissue ingrowth surface and a distal portion having a nonporous outer surface.

5. The prosthesis as set forth in claim 1 wherein the combined thickness of the PMMA sleeve and the metal sheath is between 1 and 3 mm.

6. A method of forming a prosthetic hip implant comprising:
providing a prosthetic hip implant having tapered metal stem portion and a neck portion;
covering the stem portion with a polymethylmethacrylate (PMMA) sleeve; and
bonding the PMMA sleeve to an inner surface of a metal sheath having a porous proximal outer surface.

7. The method as set forth in claim 6 wherein the metal sheath is formed by a process selected from the group consisting of sputtering and selective laser sintering.

8. The method as set forth in claim 6 wherein the PMMA sleeve is applied to the stem portion by spraying or dipping.

9. The method as set forth in claim 6 wherein the PMMA sleeve is preformed and has an inner surface matching an outer surface of the stem.

10. The method as set forth in claim 9 further comprising polishing the stem outer surface prior to insertion into the preformed sleeve.

11. The method as set forth in claim 9 wherein the metal sheath is preformed and has an inner surface matching an outer surface of the PMMA sleeve.

12. The method as set forth in claim 9 further comprising utilizing the stem portion to form the PMMA sleeve inner surface.

13. The method as set forth in claim 6 wherein the metal sheath has an outer distal portion which is non-porous.
